(19) United States
(12) Patent Application Publication
(10) Pub. No.: US 2003/0036668 A1
(43) Pub. Date: Feb. 20, 2003

(54) Fluorination method

(76) Inventors: Roy Dennis Bowden, Lea Town Presto PR4 Orz (GB); John Steward Moilliet, Lea Town, Preston PR4 Orz (GB)

Correspondence Address: CROWELL & MORING LLP, INTELLECTUAL PROPERTY GROUP, P.O. BOX 14300, WASHINGTON, DC, 20044-4300, US

(21) Appl. No.: 10/009,949

(22) PCT Filed: Apr. 11, 2001
(86) PCT No.: PCT/GB01/01689

(30) Foreign Application Priority Data
Apr. 14, 2000 (GB) 0009156.1

(51) Int. Cl. C07C 17/08; C07C 45/63

(52) U.S. Cl. 570/161; 568/323

(57) ABSTRACT

Novel techniques are disclosed for the direct fluoridation of organic compounds by treatment with fluorine gas in a reaction mixture comprising hydrofluoric acid and at least one of water and formic acid. The techniques are particularly applicable to aromatic organic compounds and beta-dicarbonyl compounds. Increased efficiency of reaction is observed, together with improvements in yield and purity of product, with especially beneficial effects being apparent at low temperatures.

The present invention relates to novel techniques for the fluorination of organic compounds. More particularly, the invention provides a method for the fluorination of aromatic organic compounds and beta-dicarbonyl compounds which involves the use of elemental fluorine as the fluorinating agent.

Many processes for the fluorination of organic compounds are well known from the prior art. Specifically, various processes wherein fluorine gas is used as the fluorinating agent for aromatic compounds have been disclosed, but decomposition has often resulted as a consequence of the strong oxidising properties of fluorine. Thus, an alternative method which involves the dilution of the fluorine gas with an inert gas, such as nitrogen, was disclosed in EP-A-512715 and this allowed the introduction of a single fluorine atom into the aromatic compound whilst achieving a good yield. However, the preparation of polyfluoroaromatic compounds required the use of more severe conditions with a consequent increase in the formation of decomposition products.

The use of acetonitrile as an inert solvent for direct fluorination has been widely reported, but is found to be inconvenient in view of the low temperatures required and the tendency towards the formation of tar and toxic by-products.

Several examples of the use of acidic solvents are also available. EP-A-566268 describes the treatment of 2,4-difluorobenzoic acid with fluorine and nitrogen gases in trifluoroacetic acid to give high yields of the corresponding 2,4,5- and 2,3,4-trifluoro derivatives, whilst various references, including EP-B-734363, mention the use of acids including concentrated sulphuric acid, oleum and formic acid in fluorination reactions which permit the selective introduction of one or more fluorine atoms into aromatic compounds at convenient reaction temperatures, whilst providing a good overall yield.

The possibility of utilising fluorinating systems for organic compounds wherein the reaction medium comprises hydrofluoric acid has also been considered. Thus, for example, PCT application WO 97/35824 describes a process for the direct fluorination of dicarbonyl compounds in the presence of one or more acids selected from a group which includes hydrofluoric acid, sulphuric acid and trifluoroacetic acid; the process is capable of providing high yields and high selectivity. In an earlier disclosure, EP-A-18606 teaches the conversion of salicylaldehyde to its 5-fluoro derivative by direct fluorination with fluorine gas, diluted with nitrogen, in anhydrous hydrofluoric acid.

However, despite the wide range of literature disclosures relating to direct fluorination techniques, it is still the case that many fluorination reactions suffer from unwanted side-reactions, with the consequence that poor yields of products contaminated with tarry by-products are often obtained. Consequently, it is an object of the present invention to provide a convenient process for the direct fluorination of aromatic and dicarbonyl compounds which results in the formation of high yields of the desired products in a form which is free from unwanted by-products and, in particular, which is not contaminated by tarry residues.

During the course of studies of the direct fluorination of various aromatic organic compounds and dicarbonyl derivatives in mixtures of formic acid and water, the present inventors have found that the efficiency of reaction increases as the reaction proceeds. Coincident with this increase in reaction efficiency is the formation of hydrogen fluoride in the reaction mixture, resulting from the reaction between the fluorine gas and the solvent. Further studies of this phenomenon have shown that optimisation of the fluorination reaction can be achieved by adjustment of the levels of hydrogen fluoride and water in the reaction mixture, such that the yield, conversion and fluorine efficiency are all increased, whilst the amount of tar in the final product can be minimised.

Thus, according to the present invention, there is provided a process for the direct fluorination of an organic compound which comprises treating a reaction mixture comprising the organic compound and hydrofluoric acid, containing at least one of water and formic acid, with fluorine gas. Optionally, the reaction mixture may comprise hydrofluoric acid, water and formic acid.

Specifically, the organic compound comprises an aromatic compound or a beta-dicarbonyl derivative and the reaction solvent may be prepared by mixing together 98% formic acid, 60% hydrogen fluoride, anhydrous hydrogen fluoride and, if necessary, subsequently adding water to give the desired proportions of HF, HCOOH and water.

Generally, with aromatic compounds, it is found that solvent compositions comprising formic acid and water provide improved fluorine efficiency and increased conversion when compared with compositions comprising only formic acid, whilst further improvements in conversion levels, and a reduction in tar, result from the use of mixtures of formic acid and hydrogen fluoride. Compositions comprising formic acid, hydrogen fluoride and water also show similar increases in conversion, fluorine efficiency and yield. Particularly advantageous results are achieved with aromatic compounds when the solvent composition comprises 10-25% hydrogen fluoride, 2-20% water and 30-70% formic acid.

Efficient reactions have, however, also been observed when using levels of hydrogen fluoride of 60% or, more advantageously, 80%, with water providing the balance of the solvent, although it appears that the most satisfactory results are achieved when using solvent compositions containing 90-100% hydrogen fluoride, the balance being water.

Conversely, solvents containing 70% formic acid have also proved to be especially effective in certain circumstances. Specifically, the fluorination of 4-nitrotoluene has been found to proceed efficiently using a solvent mixture comprising 50-70% formic acid, 20-30% hydrogen fluoride and 2-20% water.

The reactions are generally carried out at a temperature of between −30° and +30° C., with particularly advantageous results being obtained when the temperature is controlled in the range of from −15° to +10° C.

Further examples of aromatic compounds which may advantageously be fluorinated according to the method of the present invention include alkylbenzenes, such as derivatives of toluene and ethylbenzene, and substituted aromatic compounds, for example 4-chloronitrobenzene.

Various beta-dicarbonyl compounds may also be effectively fluorinated according to the method of the present invention. Typical examples of such compounds include ketoesters and, in particular, diketones. In the latter case, advantages are most apparent when using levels of hydrogen fluoride in the region of 60%, and significant improvements in yield and fluorine efficiency may be achieved.

In addition to the advantageous features of the present method which are evident in terms of efficiency of reaction, and increase in yield and purity of product, there are also economic benefits which are readily apparent. These benefits accrue as a result of the fact that the hydrogen fluoride which, in the form of hydrofluoric acid, serves as a solvent for the reaction, is recoverable and, consequently, may be used for further reactions; by way of contrast, this advantage does not accrue in the case of solvents such as formic acid or sulphuric acid.

The present inventors have carried out a series of experiments, by means of which it has been possible to establish the optimum reaction conditions for various organic compounds falling within the scope of the present invention. The following examples are, therefore, illustrative of the invention, without placing any limitation on the scope thereof:

### EXAMPLES

### Example 1.1

Reaction of Fluorine with 4-nitrotoluene

13.7 g (0.1 mole) 4-nitrotoluene were put into the reactor and 58.2 g of 60% HF were added. This was cooled in an ice bath while 61.7 g of anhydrous HF, which had previously been trapped in a cooled vessel from a cylinder of HF, were added. This represents an HF strength of 80%.

The reactor was cooled to 0° C. and a mixture of fluorine, at 10% in nitrogen, was passed through the reaction solution until 0.12 moles of fluorine had been reacted. The solution was cooled and vigorously agitated during this addition. The fluorine was switched off and the nitrogen continued for a further 10 minutes.

The reaction solution was poured out onto 200 g of ice and extracted three times with methylene chloride (3×25 ml). After drying over magnesium sulfate the solvent was removed and the residue (15.6 g) was distilled on a Kuegel-Rohr apparatus at 0.1 mbar. The product (13.0 g) which distilled over at approximately 110° C. was analysed by gas chromatography and shown to contain 9.6 g of 2-fluoro 4-nitrotoluene and 1.4 g of starting material. There was 1.4 g of residual material.

This represents a yield of 73% at a conversion of 88% and a fluorine efficiency of 52%. The weight of tar was 12% of the consumed starting material.

### Comparative Example 1.2

Details as above but, using 60% HF (100 ml, 116.7 g), 0.3 moles of fluorine were added over 12 hours.

After work up the crude weight was 14.8 g which were distilled to give 10.0 g leaving 4.6 g of tar. GC analysis showed that it contained 4.0 g 2-fluoro 4-nitrotoluene and 4.6 g of starting material as well as a number of di- and tri-fluorinated products. This represents a yield of 41% at a conversion of 65% and a fluorine efficiency 9%. The weight of tar was 52% of the consumed starting material.

It is therefore clear that 80% HF is better than 60%, so it would seem that the amount of water has to be defined; further details are given in Table 1A.

TABLE 1A

| | Solvent | | | | | | | |
|---|---|---|---|---|---|---|---|---|
| | Formic | | | F₂ | | | F₂ | |
| Example | acid | HF | Water | equivs | Yield | Conv. | eff. | Tar |
| 1.1 | — | 80 | 20 | 1.2 | 73 | 88 | 52 | 12 |
| 1.2 | — | 60 | 40 | 2.9 | 41 | 65 | 9 | 52 |
| 1.3 | 98 | — | 2 | 2.2 | 53 | 9 | 2 | 33 |
| 1.9 | 80 | — | 20 | 3 | 55 | 86 | 16 | 33 |
| 1.13 | 80 | 18 | 2 | 5.5 | 73 | 76 | 10 | 19 |

Further experiments were, therefore, carried out using different levels of formic acid, water and HF in order to define the optimum conditions. The results are summarised in Table 1B.

In each case, the procedure involved preparation of the reaction solvent by mixing together in the correct proportions, the following materials:

a. 98% formic acid
b. 60% hydrogen fluoride
c. anhydrous hydrogen fluoride
d. water

The substrate was then dissolved in 100 ml of the solvent and stirred at the right temperature while the fluorine/nitrogen mixture was passed through. After the required amount of fluorine had been added the gas mixture was switched off and passage of pure nitrogen continued for a further ten minutes. The contents of the reactor were poured out onto 200 g of ice and extracted with 3×50 ml of methylene chloride. After drying over magnesium sulphate the solvent was removed by rotary evaporation and the residue distilled under reduced pressure in a Kuegel-Rohr apparatus. The distillate was analysed by gas chromatography.

TABLE 1B

| | | SOLVENT | | | F₂ added | | | F₂ | |
|---|---|---|---|---|---|---|---|---|---|
| Example | SUBSTRATE | Formic acid | Water | HF | Equivalents | Product Yield | Conversion | Efficiency | Tar |
| 1.3 | 4-nitrotoluene | 98 | 2 | 0 | 2.2 | 53 | 9 | 2 | 33 |
| 1.4 | do. | 98 | 2 | 0 | 2.6 | 56 | 19 | 4 | 27 |
| 1.5 | do. | 98 | 2 | 0 | 3.5 | 64 | 32 | 6 | 27 |
| 1.6 | do. | 98 | 2 | 0 | 5 | 70 | 56 | 8 | 27 |
| 1.7 | do. | 98 | 2 | 0 | 6.5 | 70 | 71 | 7 | 24 |
| 1.8 | do. | 90 | 10 | 0 | 3 | 65 | 38 | 9 | 35 |
| 1.9 | do. | 80 | 20 | 0 | 3 | 55 | 86 | 16 | 33 |
| 1.10 | do. | 60 | 40 | 0 | 3 | 41 | 77 | 10 | 46 |
| 1.11 | do. | 88 | 2 | 10 | 3 | 63 | 12 | 2 | 25 |
| 1.12 | do. | 80 | 2 | 18 | 3 | 67 | 36 | 8 | 12 |
| 1.13 | do. | 80 | 2 | 18 | 5.5 | 73 | 76 | 10 | 19 |
| 1.14 | do. | 70 | 1 | 29 | 5 | 68 | 75 | 10 | 23 |
| 1.15 | do. | 80 | 10 | 10 | 3 | 65 | 26 | 6 | 26 |
| 1.16 | do. | 60 | 20 | 20 | 3 | 70 | 65 | 15 | 19 |
| 1.17 | do. | 60 | 10 | 30 | 3 | 69 | 80 | 16 | 19 |
| 1.18 | do. | 70 | 20 | 10 | 3.1 | 69 | 64 | 14 | 24 |

Definitions:

$$\begin{matrix}Yield = \frac{weight of product}{\begin{matrix}weight of product which would have been \\ formed from consumed starting material\end{matrix} } \\ Conversion = \frac{weight of starting material consumed}{weight of starting material} \\ Fluorine efficiency = \frac{weight of fluorine in product}{50 \% of the weight of fluorine fed into reactor} \\ \% Tar = \frac{weight of tar}{weight of starting material consumed}\end{matrix}$$

### Example 2

Reaction of Flourine with 4′-methoxyacetophenone

4′methoxyacetophenone (15.0 g) was placed in the reactor and the solvent mixture, prepared as in Example 1, was added. The same procedure was adopted as in Example 1.1 outlined above and the results are summarised in Table 2 below.

TABLE 2

| | Solvent | | | | | | | |
|---|---|---|---|---|---|---|---|---|
| | Formic | | | F₂ | | | F₂ | |
| Example | acid | HF | Water | equivs | Yield | Conv. | eff. | Tar |
| 2.1 | 98 | — | 2 | | 55 | 76 | | 18 |
| 2.2 | 80 | — | 20 | | 52 | 85 | | 10 |
| 2.3 | — | 60 | 40 | 1 | 51 | 59 | 33 | 36 |
| 2.4 | — | 80 | 20 | 1.5 | 46 | 81 | 24 | 24 |

### Example 3

Fluorination of 2-nitrotoluene

The reaction was carried out as described in Example 1.1 and the results are summarised in Table 3.

TABLE 3

| | Solvent | | | | | | | |
|---|---|---|---|---|---|---|---|---|
| | Formic | | | F₂ | | | F₂ | |
| Example | acid | HF | Water | equivs | Yield | Conv. | eff. | Tar |
| 3.1 | 98 | | 2 | 2.7 | 33 | 14 | 2 | 32 |
| 3.2 | — | 60 | 40 | 1.3 | 42 | 74 | 23 | 41 |
| 3.3 | — | 80 | 20 | 1.3 | 60 | 85 | 38 | 21 |

### Example 4

Fluorination of 4-chloro nitrobenzene

The reaction was carried out as in Example 1.1. The results are shown in Table 4.

TABLE 4

| | Solvent | | | | | | | |
|---|---|---|---|---|---|---|---|---|
| | Formic | | | F₂ | | | F₂ | |
| Example | acid | HF | Water | equivs | Yield | Conv. | eff. | Tar |
| 4.1 | 98 | — | 2 | 2.5 | 0 | 5 | 0 | 6 |
| 4.2 | — | 60 | 40 | 3.8 | 38 | 60 | 5 | 54 |
| 4.3 | — | 80 | 20 | 1.6 | 62 | 84 | 25 | 18 |

### Example 5

Reaction of Fluorine with methyl 3-oxopentanoate

Methyl 3-oxopentanoate (13.0 g; 0.1 mole) was dissolved in 60% HF (100 ml) and cooled with stirring to 3° C. A gaseous mixture of fluorine (10%) in nitrogen was passed for 8 hours at 100 ml/min while maintaining both the stirring and cooling.

After this time the fluorine was switched off and pure nitrogen passed for a further 10 minutes.

The contents of the reactor were poured onto 200 g ice and extracted with 3×50 ml methylene chloride. After drying over magnesium sulphate the solvent was removed by rotary evaporation and 11.3 g of residue remained. This was distilled on a Kuegel-Rohr apparatus at 12 mbar and up to 125° C. to give 7.0 g of product while 0.8 g of tar was left behind.

GC analysis of the distilled product indicated it contained 6.5 g of 2-fluoro-3-oxopentanoate and 0.2 g of starting material. This represents a yield of 45% at a conversion of 99%.

Several different solvent mixtures were investigated and the results are shown in Table 5.

TABLE 5

| | Solvent | | | | | | | |
|---|---|---|---|---|---|---|---|---|
| | Formic | | | F₂ | | | F₂ | |
| Example | acid | HF | Water | equivs | Yield | Conv. | eff. | Tar |
| 5.1 | 98 | — | 2 | 3.8 | 88 | 55 | 12 | 3 |
| 5.2 | 80 | — | 20 | 3.7 | 84 | 71 | 16 | 2 |
| 5.3 | 80 | 18 | 2 | 3.8 | 71 | 68 | 12 | 7 |
| 5.4 | — | 60 | 40 | 1.6 | 45 | 99 | 27 | 5 |
| 5.5 | — | 80 | 20 | 1.4 | 62 | 99 | 42 | 2 |

### Example 6

Reaction of Fluorine with Pentan-2,4-dione

The same procedure was carried out as in Example 5, except that the crude product was distilled at atmospheric pressure in a conventional distillation apparatus. 10.3 g of crude gave 4.2 g of 3-fluoropentanedione and 2.3 g of 3,3-difluoropentanedione, leaving 0.2 g of tarry residue. This is a yield of 41% monofluoro- (64% of mono and difluoro- products) at 80% conversion.

Various different solvent systems were tested and the results are detailed in Table 6. Yields are quoted for monofluoro- drerivatives and (in parentheses) for total monofluoro-/difluoro- products.

TABLE 6

| | Solvent | | | | | | | |
|---|---|---|---|---|---|---|---|---|
| | Formic | | | F₂ | | | F₂ | |
| Example | acid | HF | Water | equivs | Yield | Conv. | eff. | Tar |
| 6.1 | 98 | — | 2 | 6.2 | 39(46) | 100 | 8 | 5 |
| 6.2 | 80 | — | 20 | 2.7 | 54(58) | 100 | 31 | 11 |
| 6.3 | 80 | 18 | 2 | 1.7 | 61(66) | 85 | 31 | 6 |
| 6.4 | — | 60 | 40 | 1.3 | 64(68) | 100 | 58 | 6 |
| 6.5 | — | 80 | 20 | 1.0 | 41(64) | 88 | 67 | 2 |

1. A process for the direct fluorination of an organic compound which comprises treating a reaction mixture comprising the organic compound and hydrofluoric acid, containing at least one of water and formic acid, with fluorine gas.

2. A process as defined in claim 1, wherein the reaction mixture comprises hydrofluoric acid, water and formic acid.

3. A process according to claim 1 or claim 2, wherein the organic compound is an aromatic compound or a beta-dicarbonyl compound.

4. A process according to any of claims 1-3, wherein the reaction mixture contains a solvent comprising 25-40% hydrogen fluoride, 2-20% water and 30-80% formic acid.

5. A process according to any of claims 1-3, wherein the reaction mixture contains a solvent comprising greater than 60% hydrogen fluoride.

6. A process according to claim 5 wherein the reaction mixture contains a solvent comprising 90-100% hydrogen fluoride and 0-10% water.

7. A process according to any of claims 1-3, wherein the reaction mixture comprises a solvent containing at least 80% formic acid.

8. A process according to claim 3, wherein the solvent comprises 50-70% formic acid, 20-30% hydrogen fluoride and 2-20% water.

9. A process according to any of the preceding claims, wherein the organic compound is an alkylbenzene or a substituted aromatic ketone.

10. A process according to any of the preceding claims, wherein the organic compound is toluene, nitrotoluene, ethylbenzene or 4-chloronitrobenzene.

11. A process according to any of claims 1 to 8, wherein the organic compound is a ketoester or a diketone.

12. A process according to claim 11, wherein the reaction mixture includes a solvent comprising 60% hydrogen fluoride.

13. A process according to any of the preceding claims wherein the reaction temperature is in the range of from −30° to +30° C.

14. A process as defined in claim 13 wherein the reaction temperature is in the range of from −15° to +10° C.
